(19) United States
(12) Patent Application Publication
(10) Pub. No.: US 2016/0100809 A1
(43) Pub. Date: Apr. 14, 2016

(54) PARAMETER MODULES INCLUDING DETACHABLE PROCESSING DEVICES AND METHODS OF USING THE SAME

(71) Applicant: Xhale, Inc., Gainesville, FL (US)

(72) Inventor: Sean Cohen, Gainesville, FL (US)

(21) Appl. No.: 14/878,885

(22) Filed: Oct. 8, 2015

Related U.S. Application Data
(60) Provisional application No. 62/062,280, filed on Oct. 10, 2014.

(51) Int. Cl. A61B 5/00; A61B 5/1455

(52) U.S. Cl. CPC A61B 5/7445; A61B 5/14551; A61B 5/7271; A61B 2560/0443

(57) ABSTRACT

Provided according to embodiments of the present invention are multiparameter monitors that include a primary screen that displays signals or parameters provided by at least one parameter module; and a parameter module that includes a detachable processing device.

### CROSS-REFERENCE TO RELATED APPLICATIONS

This application claims the benefit of U.S. Provisional Application No. 62/062,280, filed Oct. 10, 2014, the contents of which are incorporated herein by reference in their entirety.

### FIELD OF THE INVENTION

The present invention relates to biological sensors, and in particular, to photoplethysmography sensors. The present invention also relates to methods and devices for monitoring for monitoring oxygen saturation and other cardiopulmonary parameters.

### BACKGROUND OF THE INVENTION

Photoplethysmography, or “PPG”, is an optical technique for detecting blood volume changes in a tissue. In this technique, one or more emitters are used to direct light at a tissue and one or more detectors are used to detect the light that is transmitted through the tissue (“transmissive PPG”) or reflected by the tissue (“reflectance PPG”). The volume of blood, or perfusion, of the tissue affects the amount of light that is transmitted or reflected. Thus, the PPG signal may vary with changes in the perfusion of the tissue.

In many hospital units, multiparameter monitors are used to display a variety of different physiological parameters. Such monitors typically have a rack or docking station for different modules to be inserted, and the modules, once connected, can receive signals from a physiological sensor and then display the signals or physiological parameters calculated therefrom on the multiparameter monitor.

### SUMMARY OF EMBODIMENTS OF THE INVENTION

Provided according to embodiments of the present invention are multiparameter monitors that include a primary screen that displays signals or parameters provided by at least one parameter module; and at least one parameter module that includes a detachable processing device. In some embodiments, the at least one parameter module may in communication with a physiological sensor, such a PPG sensor. The parameter module including the detachable processing device receives signals from the physiological sensor and processes the signals; and when the detachable processing device is connected to the parameter module, the primary screen displays the signals and/or parameters calculated by the parameter module comprising the detachable processing device.

Furthermore, in some embodiments, when the detachable processing device is disconnected from the parameter module, the auxiliary screen may display the signals and/or parameters calculated by the parameter module that includes the detachable processing device. In addition, in some embodiments, the detachable processing device switches from displaying the signals and/or parameters on the primary screen to the auxiliary screen automatically upon disconnecting the detachable processing device from the parameter module.

Also provided according to embodiments of the invention are parameter modules for insertion into a multiparameter monitor rack, wherein the parameter modules include a detachable processing device.

Further provided according to embodiments of the invention, are methods of monitoring an individual that include attaching a physiological sensor to the individual; and connecting the physiological sensor to a multiparameter monitor, wherein the multiparameter monitor includes a primary screen that displays signals or parameters provided by at least one parameter module, and a parameter module including a detachable processing device. In some embodiments, the detachable processing device receives signals from the physiological sensor and processes the signals. When the detachable processing device is connected to the parameter module, the primary screen displays the signals and/or parameters calculated by the parameter module including the detachable processing device, and when the detachable processing device is disconnected from the parameter module, an auxiliary screen on the detachable processing device displays the signals and/or parameters calculated by the parameter module including the detachable processing device.

### BRIEF DESCRIPTION OF THE DRAWINGS

FIG. 1 shows a multiparameter monitor according to some embodiments of the invention.

FIG. 2 shows a parameter module comprising a detachable processing device according to some embodiments of the invention.

FIG. 3A shows a detachable processing device and module base according to embodiments of the invention.

FIG. 3B shows the detachable processing device and module base in FIG. 3A after the detachable processing device has been ejected from the module base.

### DETAILED DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers refer to like elements throughout the specification.

It will be understood that, although the terms first, second, etc. (or primary, secondary, etc.) may be used herein to describe various elements, these elements should not be limited by these terms. These terms are only used to distinguish one element from another. Thus, a first element discussed below could be termed a second element without departing from the teachings of the present invention.

Patients are often transported between different hospital units. For example, patients transition from acute care units to general care floors, and are transported from their primary location for imaging or for medical tests. While patients are often monitored closely at each hospital unit, they may be unmonitored while in transport, or if a transport monitor is used, they may have to unplug from one monitor to another and continuous monitoring may be disrupted. The present invention is directed to parameter modules (for multiparameter monitors) that have detachable processing devices, which may allow for patients to be continuously monitored during transport.

Provided according to embodiments of the present invention are methods, monitors and devices for monitoring a physiological state of an individual. The present invention describes embodiments wherein a parameter module of a multiparameter monitor includes a detachable processing device. Such a processing device may be connected to the parameter module in some cases, and signals or parameters calculated therefrom (or other data) may be displayed on the screen of the multiparameter monitor. However, in other cases, such as when an individual is being transported from one location (e.g., of a hospital) to another, the detachable processing device may be disconnected from the parameter module, and may continue monitoring the individual using only the detachable processing device, which is portable and can travel with the individual. The detachable processing device can also later be connected to the original parameter module or another compatible parameter module at a different location. Thus, the individual can have continuity of care while being transported through the hospital or while in a any situation whereby the use of the multiparameter monitor is not possible or not desired.

### The Individual

The systems and methods described herein are used for monitoring the physiological state of an individual. As used herein, an individual, also referred to as a patient, includes any mammal, including humans of any age. The individual may be monitored in any care setting including, but not limited to, hospitals (e.g., operating room (OR), intensive care unit (ICU), general care floors, or during transport therein); nursing homes, medical offices, medical transport, and homes. Furthermore, the methods, devices and systems described herein may be useful for both spontaneously breathing patients and mechanically ventilated patients.

### The Multiparameter Monitor Including Parameter Modules

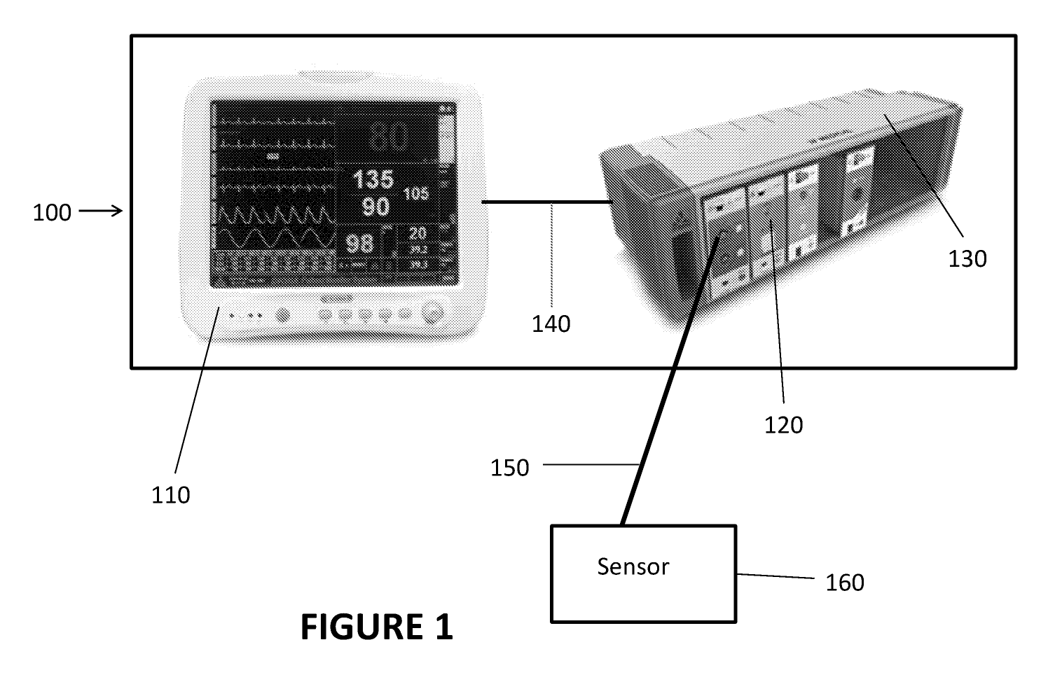

Provided according to embodiments of the invention are multiparameter monitors that include a primary screen that displays signals or parameters provided by at least one parameter module; and a parameter module that includes a detachable processing device. Referring to FIG. 1, a multiparameter monitor 100 includes a screen 110 and at least one parameter module 120. The at least one parameter module 120 is typically contained in a module rack 130, and the at least one parameter module 120 is in communication 140 with the screen 110 which is configured to display signals or parameters processed or transmitted by the at least one parameter module 120. The multiparameter monitor 100 can thus display different parameters on the screen 110 based on the parameter parameter modules 120 included in the module rack 130, and thus is configurable according to the needs of the particular medical monitoring setting. The at least one parameter module 120 is also in communication 150 with at least one physiological sensor 160. The at least one physiological sensor 160 obtains physiological signals from the individual and transmits the signals to the at least one parameter module 120. The at least one parameter module 120 processes the signals to provide a processed signal or calculate a physiological parameter and communicates 140 the signals or physiological parameters to the screen 110 for display to medical personnel. FIG. 1 does not show a parameter module having a detachable processing device, but only shows the typical parameter modules that are currently used in medical care settings.

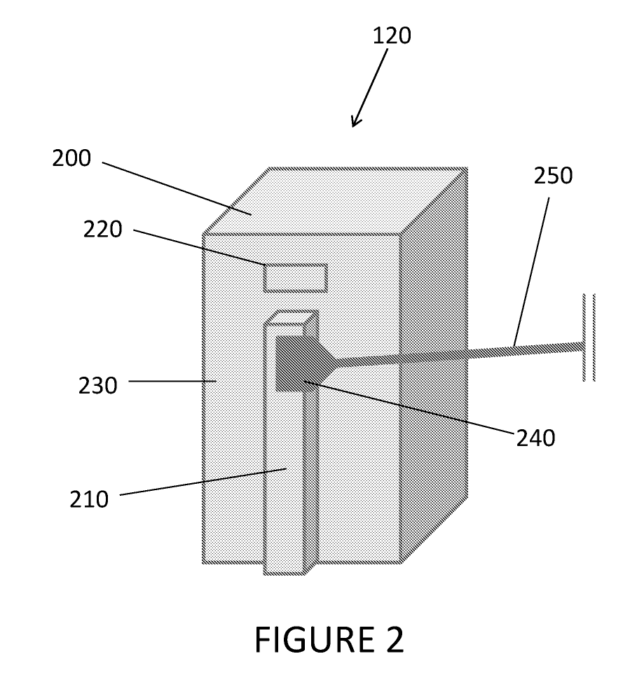

FIG. 2 provides an illustration of a parameter module comprising a detachable processing device according to some embodiments of the invention. The parameter module 120 includes a module base 200 in which the detachable processing device 210 may be inserted or to which the detachable processing device 210 may be otherwise be attached. In the embodiment shown in FIG. 2, the detachable processing device 210 is inserted into the module base 200 and may be ejected by an eject button 220. In some embodiments, in lieu of inserting into the parameter base, the detachable processing device 210 may attach to the face 230 of the parameter base 200 or to a recess (not shown) in the module base 200. In some embodiments of the invention, the detachable processing device 210 includes a connector 240 for attaching a wire or cable 250 of a physiological sensor (not shown). The connector 240 and wire or cable 250 can be of any suitable shape, size or configuration. Furthermore, additional connectors for communicating with additional physiological sensors may also be included.

The module base 200 may holds the detachable processing device 210 and connects it to the multiparameter monitor screen, but in some embodiments, the module base 200 also may charge the detachable processing device 210. In some embodiments, the module base 200 may have additional processing capabilities. For example, in some embodiments, when the detachable processing device 210 is connected to the module base 200, certain physiological signals or parameters may be measured by the detachable processing device 210 and/or a processor in the processor in the module base 200. Then, when the detachable processing device 210 is disconnected, a subset of physiological signals or parameters may be monitored by the detachable processing device 210. Thus, the parameter module 120 may process additional information when the detachable processing device 210 is connected than the detachable processing device 210 can achieve while disconnected. However, in some embodiments, no additional processing power is included in the module base 200 other than that needed to transmit and display the signal and parameter information from the detachable processing device 210 to the multiparameter monitor screen.

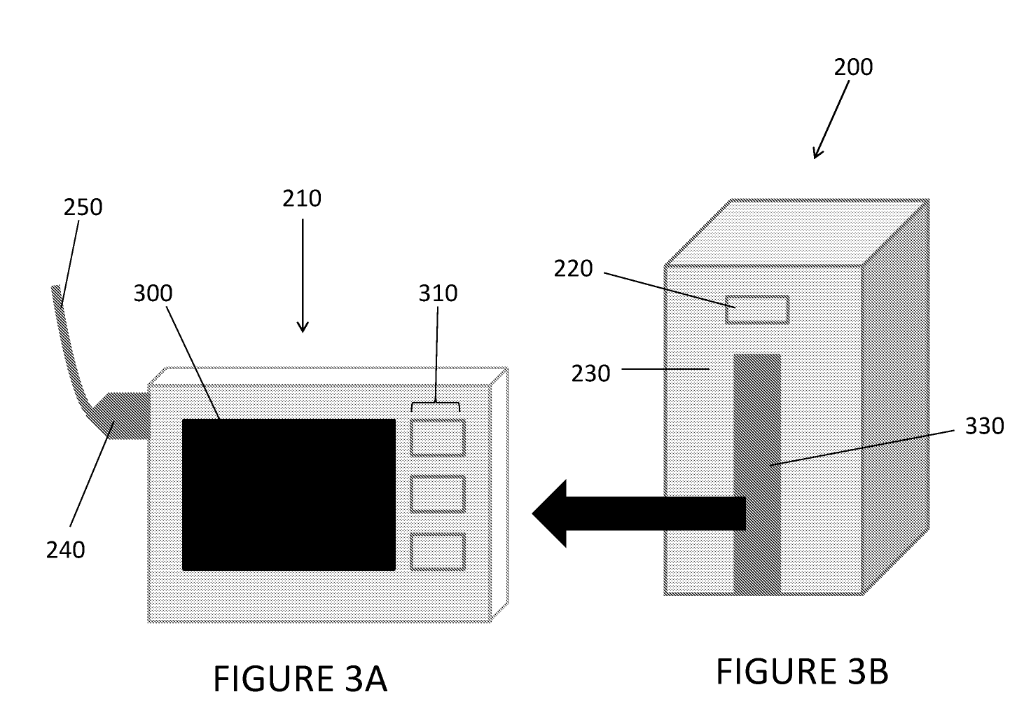

FIGS. 3A and 3B show the detachable processing device 210 (FIG. 3A) and the module base 200 (FIG. 3B) after the detachable processing device 210 in FIG. 2 is ejected from the module base 200. In some embodiments, the detachable processing device 210 may include an auxiliary screen 300 for display of physiological signals and/or parameters and optionally one or more user navigation buttons 310 that may be used, for example, to view particular information, to respond to alarms, or turn the power on or off. Once the detachable processing device 210 is removed from the module base 200, a device bay 300 may be exposed. In some cases, the detachable processing device 210 is attached to the facing 230 of the module base 200 so that no device bay 300 is exposed when the detachable processing device 210 is removed. In some cases, however, a recess in the module base 200 may remain when the detachable processing device 210 is removed. In some embodiments of the invention, the detachable processing device 210 may be interchangeably attached or inserted in the device bay 300 of a number of different module bases 200. As such, the detachable processing device 210 may connect and be used at a number of different locations.

In some embodiments of the invention, when the detachable processing device is connected to the parameter module, the primary screen (multiparameter monitor screen) displays the signals and/or parameters calculated by the parameter module that includes the detachable processing device, but when the detachable processing device is disconnected from the parameter module, the auxiliary screen displays the signals and/or parameters. In some cases, the detachable processing device switches from displaying the signals and/or parameters on the primary screen to the auxiliary screen automatically upon disconnecting the detachable processing device from the parameter module.

### The PPG Sensor and Optional Auxiliary Respiration Sensor

The systems and methods described herein use at least one processing device to process and analyze physiological signals. Any suitable physiological signal may be used, but in some embodiments, the physiological signals are photoplethysmography (PPG) signals. The PPG signals processed by the signal processing devices may be obtained in any suitable manner and at any suitable physiological site. However, in some embodiments, the PPG signal will be obtained at a site at or on the head of the individual (also referred to herein as a “central source site”). Such sites include, but not are limited to, the ear, nose (e.g., the nasal alar, nasal septum or nasal columella), forehead, cheek, lip, ophthalmic artery, and pre-auricular or post-auricular sites.

The PPG signals may be obtained by securing at least one PPG sensor (also referred to as a PPG probe) to the individual. The term “secure” means to attach sufficiently to the tissue site to allow for a suitable PPG signal to be generated. In some cases, the sensor is configured to secure onto a tissue site such that no additional support is necessary to allow for a suitable PPG signal to be reliably generated. However, in some cases, the sensor may be secured with the aid of an external support, for example, an additional structural support, a wire or cord, or an adhesive product such as tape. Such supports may be desirable to stabilize the sensor to prevent against signal loss, for example, due to the patient's movement, or due to movement (e.g., jostling, pulling, pushing) of the sensor or a cable attached thereto.

The PPG sensors include one or more components that emit light, and such components will be referred to herein as “emitters.” As used herein, the term “light” is used generically to refer to electromagnetic radiation, and so the term includes, for example, visible, infrared and ultraviolet radiation. Any suitable type of emitter may be used, but in some embodiments, the emitter is a light-emitting diode (LED). In particular embodiments, a first emitter emits light at a first wavelength, and a second emitter emits light at a second wavelength. In some cases, a single emitter may emit light at a first wavelength and a second wavelength. One or more photodetectors, also referred to as “detectors”, are also included in the PPG sensor. The detector is configured to detect light from an emitter, and this detected light generates a PPG signal. Any suitable photodetector may be used. However, examples of photodetectors include photodiodes, photoresistors, phototransistors, light to digital converters, and the like. The PPG signals are monitored over time and the PPG signals generated by the photodetector(s) may be referred to as “PPG signals,” “a PPG signal stream,” or “a PPG waveform.”

In some embodiments of the invention, the PPG sensor may be integrated with or connected to an auxiliary respiration sensor, and the detachable processing device may process data from a PPG sensor and/or the auxiliary respiration sensor. The PPG signals may themselves be used to monitor respiration, such as described, for example, in U.S. Pat. No. 7,785,262 and U.S. Patent Application Publications 2013/0276785 and 2013/0296823, which are incorporated herein by reference in their entirety. Other methods of monitoring respiration from PPG signals are known and may be used in embodiments described herein. The auxiliary respiration sensor may be used either to monitor respiration by itself, whereby the PPG signals are only used to monitor blood oxygen saturation and/or other conventional parameters, or signals from the auxiliary respiration sensor may be used in combination with the PPG signals to monitor respiration. The respiratory data from the auxiliary respiration sensor can be compared or combined with the information from the PPG signals to provide greater certainty regarding the respiratory events or parameters or to provide additional respiratory data not available with the PPG signals. The PPG sensor and the auxiliary respiration sensor may share a signal pathway such as a cable, wire and the like, or they may be transmitted separately to a signal processing device for processing and/or analysis.

Auxiliary respiration sensors include, but are not limited to, nasal air flow sensors, nasal pressure sensors, capnometers, thermistors, acoustic sensors, differential pressure transducers, chest or abdominal bands, and the like. In some cases, both the PPG sensor(s) and the auxiliary respiration sensor(s) are situated at the nose, and in some cases, a single device or system (e.g., an array) may include both the PPG sensor(s) and the auxiliary respiration sensor(s).

In particular embodiments, the auxiliary respiration sensor may detect respiratory airflow or temperature changes at the nostril, such as with a thermistor. For example, during inspiration, a thermistor placed at the nostril detects a relative decrease in temperature compared to exhalation since, in most situations, body temperature, and therefore exhaled breath temperature, is higher than ambient temperature. Thus, detection of changes in temperature may be a suitable means to determine respiratory air flow and therefore, respiratory rate. Air flow from one or both nostrils may be monitored and, in some cases, be compared with the PPG information.

As another example, capnometry may provide a number of respiratory parameters. Such parameters may generally be reliably used for monitoring adequacy of ventilation if the patient is intubated. Unfortunately both hyper- and hypoventilation in patients may cause the capnometry results may be unreliable. However, in some cases, capnometry may be useful as an auxiliary respiration sensor to detect the respiratory airflow and, in some cases, may be used to validate the PPG-based (or other) respiratory determinations.

Other physiological sensors may be present in some embodiments of the invention. In some embodiments of the invention, the detachable processing device may process data from a PPG sensor and/or the other physiological sensor(s). For example, the PPG sensor may be integrated with or connected to physiological sensors including, but not limited to, oxygen sensors, pH sensors, blood pressure sensors, breath constituent sensors, blood constituent sensor, heart rate or activity sensors (e.g., ECG sensors) and depth of anesthesia sensors. Signals from such sensors may be transmitted and processed in addition to, or in lieu of, the signals from the auxiliary respiration sensor. Thus, in some embodiments, the first or additional signal processing devices may process signals from one or more of these additional physiological sensors in order to determine a physiological parameter or event. It is to be understood that in any embodiments described herein, the auxiliary respiration detector may be replaced with the “other physiological sensors.”

### Signal Processing Devices

The detachable processing device may include any device that can receive physiological signals and process them to determine a physiological parameter, physiological event or other measure of the physiological state of the individual. In some embodiments, the processing device(s) may also process and display the data in real time, which refers to the fact that the signals do not have to be averaged over time but the physiological parameters or events can be communicated to caregivers or the individual at the time of their occurrence or immediately thereafter.

Any suitable processing device may be used as the detachable processing device, and such devices include, for example, a general-purpose microprocessor, a digital signal processor (DSP) or application specific integrated circuit (ASIC). While the singular of such terms is used, a “signal processing device” may also include two or more signal processing devices integrated together. Such a microprocessor may be adapted to execute software, which may include an operating system and one or more applications, as part of performing the functions described herein. In electronic communication with the microprocessor may be a computer memory, such as a read-only memory (ROM), random access memory (RAM), and the like. Any suitable computer-readable media may be used in the system for data storage. Computer-readable media are capable of storing information that can be interpreted by microprocessor. This information may be data or may take the form of computer-executable instructions, such as software applications, that cause the microprocessor to perform certain functions and/or computer-implemented methods. Depending on the embodiment, such computer-readable media may include computer storage media and communication media.

Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

### Connectivity

The detachable processing device(s) may be connected to the sensor(s) in any suitable fashion, but typically they are connected via a wire, cable or other conventional means known in the art. In some embodiments, however, the signal processing device(s) may be connected to the sensors) via wireless communication, including but not limited to, Bluetooth®, WiFi, Zigbee and/or infrared technology.

In the drawings and specification, there have been disclosed embodiments of the invention and, although specific terms are employed, they are used in a generic and descriptive sense only and not for purposes of limitation, the scope of the invention being set forth in the following claims.

We claim:

1. A multiparameter monitor comprising
a primary screen that displays signals or parameters provided by at least one parameter module; and
a parameter module comprising a detachable processing device.

2. The multiparameter monitor of claim 1, wherein the detachable processing device further comprises an auxiliary screen.

3. The multiparameter monitor of claim 2, wherein a physiological sensor is in communication with the parameter module comprising the detachable processing device.

4. The multiparameter monitor of claim 3, wherein the physiological sensor comprises a photoplethysmography (PPG) sensor.

5. The multiparameter monitor of claim 4, wherein the PPG sensor is a pulse oximetry sensor.

6. The multiparameter monitor of claim 4,
wherein the parameter module comprising the detachable processing device receives signals from the physiological sensor and processes the signals; and
wherein when the detachable processing device is connected to the parameter module, the primary screen displays the signals and/or parameters calculated by the parameter module comprising the detachable processing device.

7. The multiparameter monitor of claim 6, wherein when the detachable processing device is disconnected from the parameter module, the auxiliary screen displays the signals and/or parameters calculated by the parameter module comprising the detachable processing device.

8. The multiparameter monitor of claim 7, wherein the detachable processing device connects interchangeably with a plurality of multiparameter monitors.

9. The multiparameter monitor of claim 7, wherein the detachable processing device switches from displaying the signals and/or parameters on the primary screen to the auxiliary screen automatically upon disconnecting the detachable processing device from the parameter module.

10. A parameter module for insertion into a multiparameter monitor rack, comprising a detachable processing device.

11. The parameter module of claim 10, comprising a device bay wherein the detachable processing device connects to the parameter module.

12. The parameter module of claim 11, wherein the device bay comprises a slot or recess in the parameter module.

13. The parameter module of claim 12, wherein the detachable processing device inserts into the slot in the parameter module.

14. The parameter module of claim 12, wherein the detachable processing device attaches to the recess in the parameter module.

15. The parameter module of claim 14, wherein when the detachable processing device is attached to the recess in the parameter module, at least a portion of the detachable processing device comprises the front facing of the parameter module.

16. The parameter module of claim 15, wherein the detachable processing device comprises a connector that interfaces with a physiological sensor or a cable connected thereto.

17. The parameter module of claim 16, wherein the detachable processing device comprises a screen that displays physiological signals or parameters from the physiological sensor.

18. A method for monitoring an individual, comprising
attaching a physiological sensor to the individual; and
connecting the physiological sensor to a multiparameter monitor,
wherein the multiparameter monitor comprises a primary screen that displays signals or parameters provided by at least one parameter module, and a parameter module comprising a detachable processing device.

19. The method of claim 18, wherein the
wherein the parameter module comprising the detachable processing device receives signals from the physiological sensor and processes the signals; and
wherein when the detachable processing device is connected to the parameter module, the primary screen displays the signals and/or parameters calculated by the parameter module comprising the detachable processing device, and
when the detachable processing device is disconnected from the parameter module, an auxiliary screen on the detachable processing device displays the signals and/or parameters calculated by the parameter module comprising the detachable processing device.
